(19) United States
(12) Patent Application Publication
(10) Pub. No.: US 2011/0101244 A1
(43) Pub. Date: May 5, 2011

(54) TARGET MATERIALS FOR GENERATING PROTONS AND TREATMENT APPARATUSES INCLUDING THE SAME

(75) Inventors: Moon Youn JUNG, Daejeon (KR); Nam Soo Myung, Seongnam (KR); Hyun Woo Song, Daejeon (KR); Hyeon-Bong Pyo, Daejeon (KR); Seon-Hee Park, Daejeon (KR); Gun Yong Sung, Daejeon (KR)

(73) Assignee: Electronics and Telecommunications Research Institute, Daejeon (KR)

(21) Appl. No.: 12/845,148

(22) Filed: Jul. 28, 2010

(30) Foreign Application Priority Data
Nov. 2, 2009 (KR) 10-2009-0105086
Mar. 24, 2010 (KR) 10-2010-0026418

(51) Int. Cl. A61N 5/00; B32B 15/04; C23C 16/00; H05H 1/24

(52) U.S. Cl. 250/492.1; 428/446; 428/450; 427/248.1; 427/578

(57) ABSTRACT

Provided is a treatment apparatus including a target material for generating protons. The treatment apparatus includes a cylindrical bore member having an inner space to receive a patient; a proton generating target material provided to an inner surface of the bore member; and a laser adapted to supply a laser beam to the proton generating target material so that protons are generated from the proton generating target material and projected to a tumor site of the patient. The proton generating target material includes a supporting film and a hydrogenated amorphous silicon (a-Si:H) film provided on the supporting film.

### CROSS-REFERENCE TO RELATED APPLICATIONS

This U.S. non-provisional patent application claims priority under 35 U.S.C. §119 of Korean Patent Application Nos. 10-2010-0026418, filed on Mar. 24, 2010, and 10-2009-0105086, filed on Nov. 2, 2009, the entire contents of which are hereby incorporated by reference.

### BACKGROUND OF THE INVENTION

The present invention disclosed herein relates to treatment apparatuses, and more particularly, to treatment apparatuses including target materials which generate protons.

Modern people living in a complex society are having much stress and irregular eating habits and therefore having difficulty in staying healthy. The most significant causes of death of modern people include malignant cancers and tumors. The incidence rate of cancer is socially increasing, which urgently needs national policies. To this end, various treatment methods for cancer are being spotlighted.

Cancer can be most effectively treated when found in early stage. Most of cancer treatment methods, except chemotherapy, are applied exactly to a tumor site in organs such as a brain, the breast, the ovary, and the colon.

When abnormal cells grow into a lump, it becomes easier to detect a target matter and focus on a local part, thereby enabling tumors to be removed by a surgery or destroyed by various methods including heating, cooling, irradiation and chemotherapy. However, in general, cancer spreads to nearby organs due to spread of abnormal cells from their original site. Thus, an efficient tumor treatment method needs to be developed.

### SUMMARY OF THE INVENTION

The present invention provides a target material capable of generating protons in a specific quantity required for treating a tumor.

The present invention also provides a method of manufacturing a target material that generates protons in a specific quantity required for a tumor to be treated.

The present invention also provides an apparatus for treating a tumor using protons by including a target material that generates protons in a specific quantity required for a tumor to be treated.

Objects of the present invention are not limited to those mentioned above, and other objects of the present invention will be apparently understood by those skilled in the art through the following description.

Embodiments of the present invention provide target materials for venerating protons, including a supporting film; and a hydrogenated amorphous silicon (a-Si:H) film disposed on the supporting film.

In some embodiments, the a-Si:H film may have a quantitative hydrogen atom content.

In other embodiments, the a-Si:H film may be provided on the supporting film by chemical vapor deposition (CVD).

In still other embodiments, the supporting film may include a conductive material. The conductive material may include gold (Au) or aluminum (Al).

In other embodiments of the present invention, methods of manufacturing a target material for generating protons, include preparing a supporting film; and forming a hydrogenated amorphous silicon (a-Si:H) film on the supporting film.

In some embodiments, forming the a-Si:H film may be performed by chemical vapor deposition CVD. Forming the a-Si:H film may be performed by plasma enhanced CVD (PECVD).

In other embodiments, a hydrogen atom content of the a-Si:H film may be adjusted according to conditions of the CVD. The conditions of the CVD may include composition of gas, pressure, deposition temperature, and processing time.

In still other embodiments, the supporting film may include a conductive material. The conductive material may include gold (Au) or aluminum (Al).

In other embodiments of the present invention, treatment apparatuses using protons include a cylindrical bore member having an inner space to receive a patient; a proton generating target material provided at an inner surface of the bore member; and a laser adapted to supply a laser beam to the proton generating target material so that protons are generated from the proton generating target material and projected to a tumor site of the patient, wherein the proton generating target material includes a supporting film and a hydrogenated amorphous silicon (a-Si:H) film provided on the supporting film.

In some embodiments, the a-Si:H film may have a hydrogen atom content of a specific percentage for treatment of the tumor site of the patient.

In other embodiments, the a-Si:H film may be provided on the supporting film by chemical vapor deposition (CVD).

In still other embodiments, the supporting film may include a conductive material. The conductive material may include gold (Au) or aluminum (Al).

In even other embodiments, the inner space of the bore member may be maintained under vacuum.

In yet other embodiments, the laser is a high-output laser. The laser beam may be a microwave high-output laser beam.

### BRIEF DESCRIPTION OF THE DRAWINGS

The accompanying drawings are included to provide a further understanding of the present invention, and are incorporated in and constitute a part of this specification. The drawings illustrate exemplary embodiments of the present invention and, together with the description, serve to explain principles of the present invention. In the drawings:

FIG. 1 is a sectional view schematically showing the structure of a treatment apparatus using protons, according to an embodiment of the present invention;

FIG. 2 is an enlarged sectional view of a portion A of FIG. 1, for explaining the treatment apparatus using protons according to the embodiment of the present invention; and

FIG. 3 is a sectional view for explaining a target material for generating protons according to the embodiment, and a manufacturing method thereof.

### DETAILED DESCRIPTION OF PREFERRED EMBODIMENTS

Preferred embodiments of the present invention will be described below in more detail with reference to the accompanying drawings. Advantages and features of the present invention, and implementation methods thereof will be clarified through following embodiments described with reference to the accompanying drawings. The present invention may, however, be embodied in different forms and should not be construed as limited to the embodiments set forth herein. Rather, these embodiments are provided so that this disclosure will be thorough and complete, and will fully convey the scope of the present invention to those skilled in the art.

In the following description, the technical terms are used only for explaining a specific exemplary embodiment while not limiting the present invention. The terms of a singular form may include plural forms unless referred to the contrary. The meaning of “include,” “comprise,” “including,” or “comprising,” specifies a property, a region, a fixed number, a step, a process, an element and/or a component but does not exclude other properties, regions, fixed numbers, steps, processes, elements and/or components. Since preferred embodiments are provided below, the order of the reference numerals given in the description is not limited thereto. In addition, it will be understood that when an element such as a layer, film, region, or substrate is referred to as being “on” another element, it can be directly on the other element or intervening elements may also be present.

Additionally, the embodiment in the detailed description will be described with sectional views as ideal exemplary views of the present invention. In the drawings, the dimensions of layers and regions are exaggerated for clarity of illustration. Accordingly, shapes of the exemplary views may be modified according to manufacturing techniques and/or allowable errors. Therefore, the embodiments of the present invention are not limited to the specific shape illustrated in the exemplary views, but may include other shapes that may be created according to manufacturing processes. For example, an etched region illustrated as a rectangle may have rounded or curved features. Therefore, areas exemplified in the drawings have general properties, and are used to illustrate a specific shape of a semiconductor package region. Thus, this should not be construed as limited to the scope of the present invention.

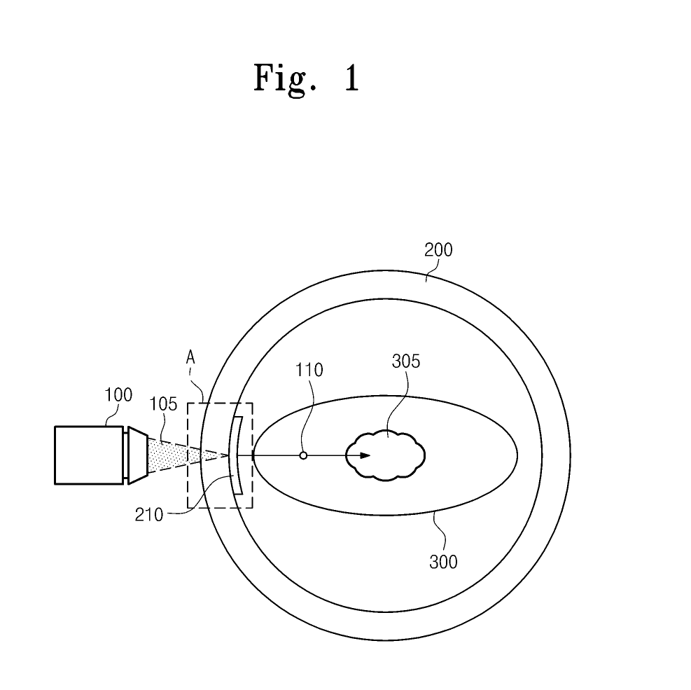

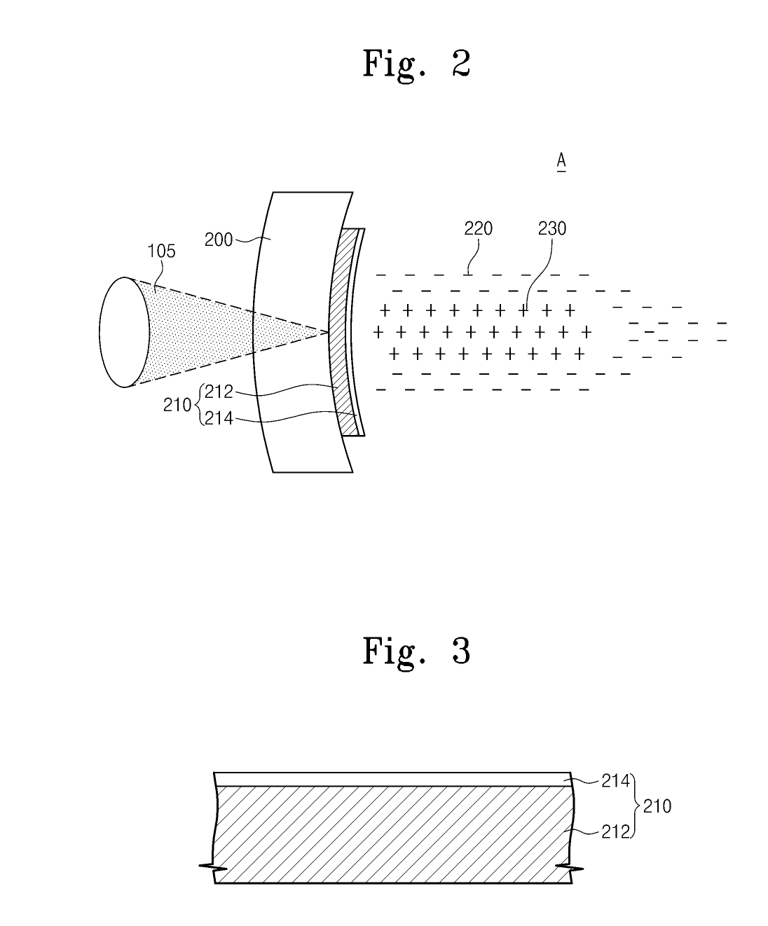

FIG. 1 is a sectional view schematically showing the structure of a treatment apparatus using protons, according to an embodiment of the present invention. FIG. 2 is an enlarged sectional view of a portion A of FIG. 1.

Referring to FIGS. 1 and 2, the treatment apparatus using protons includes a laser 100 and a target material 210 that generates protons (hereinafter, referred to as “proton generating target material 210”).

The laser 100 is adapted to project protons 110 generated from the proton generating target material 210 to a tumor site 305 of a patient 300. The laser 100 may supply a laser beam 105 to the proton generating target material 210. Here, the laser 100 may be a high-output laser and the laser beam 105 may be a microwave high-output laser beam. Accordingly, the protons 110 may be high-energy protons.

The proton generating target material 210 may generate the protons 110 using the laser beam 105 supplied from the laser 100. The proton generating target material 210 may include a metal film 212 and a hydrogen (H)-containing film 214. The metal film 212 may have a high conductivity. In other words, the metal film 212 may include gold (Au) or aluminum (Al). The H-containing film 214 may be a hydrogenated amorphous silicon (a-Si:H) film.

The treatment apparatus using protons includes an inner space adapted to receive a patient 300. The treatment apparatus may further include a cylindrical bore member 200 in which the proton generating target material 210 is disposed on an inner surface thereof. More specifically, the proton generating target material 210 may be disposed on the inner surface of the bore member 200 through the medium of a detachable adhesive material (not shown). The inner space of the bore member 200 may be maintained under vacuum and under constant-temperature and constant-humidity.

The protons 110 may be projected in a state of being set to a position corresponding to the tumor site detected by image diagnosis equipment including a magnetic resonance imaging (MRI), a computer tomography (CT), a positron emission tomography (PET), and an ultrasonic wave device.

The treatment apparatus using protons treats a tumor as follows. When the patient 300 is received in the inner space of the bore member 200, the laser beam 105 is supplied from the laser 100 to the proton generating target material 210. Accordingly, the protons 110 are generated from the proton generating target material 210 and projected into a body of the patient 300. The protons 110 projected into the body of the patient 300 collide with the tumor site 305 in the patient's body, thereby disrupting tumor cells of the tumor site 305.

More specifically, as the protons 110 disrupt the tumor cells by collision with the tumor site 305, the tumor cells may be prevented from growing or be necrotized. Here, disruption of the tumor cells by the protons 110 may include disruption of DNA double helices of the tumor cells or disruption of metabolic processes in nucleuses of the tumor cells.

Although the proton generating target material 210 is illustrated as disposed on a part of the inner surface of the bore member 200 in FIG. 1, not limited thereto, the proton generating target material 210 may cover the whole inner surface of the bore member 200.

Referring back to FIG. 2, processes of generating and projecting the protons 110 in the proton generating target material 210 of the treatment apparatus will be described in further detail.

First, when the laser beam 105 is incident to the metal film 212 of the proton generating target material 210, hydrogen atoms of the H-containing film 214 disposed on the metal film 212 are turned into a plasma state by energy of the laser beam 105, the plasma where protons 230 and electrons 220 are separated. During this, the electrons 220 are separated further than the protons 230 from the proton generating target material 210, accordingly generating an electric field by the capacitor effect between the protons 230 and the electrons 220. Since the protons 230 are accelerated toward the electrons 220 by the electric field, the protons 230 may have a sufficient energy to be projected from the outside to the tumor site 305 in the body of the patient 300.

The accelerated protons 230 may hinder growth of the tumor cells or necrotize the tumor cells by colliding with the tumor site 305 in the body of the patient 300 and disrupting the tumor cells. As a result, the tumor site 305 in the body of the patient 300 may be treated.

Since the H-containing film 214 of the proton generating target material 210 is an a-Si:H film, the H-containing film 214 may have a desired specific hydrogen atom content by varying deposition conditions thereof. That is, since the H-containing film 214 has a specific hydrogen atom content, a specific number of the protons 230 may be obtained, the protons 230 generated by the energy of the laser beam 105 incident to the metal film 212 of the proton generating target material 210 and accelerated. The H-containing film 214 may have the hydrogen atom content of several to tens of percent.

Typically, a proton generating target material is configured in a manner that a water layer is adsorbed on a metal film. In the typical proton generating target material, quantity of the water layer is varied according to the environmental conditions such as humidity, temperature, and pressure. In other words, the water layer which is a source of protons is varied in quantity by the environmental conditions in the typical proton generating target material. Therefore, the number of generated protons cannot be quantified. On the other hand, in the proton generating target material 210 according to the embodiment of the present invention, the hydrogen atoms serving as the source of the protons may be quantified since the H-containing film 214 is disposed on the metal film 212.

As described above, the treatment apparatus using protons according to the embodiment of the present invention is provided with the proton generating target material including the H-containing film having a quantitative hydrogen content, so that the protons are generated in a specific quantity required for the tumor site to be treated. Therefore, a proper quantity of the protons may be projected to the tumor site of the patient. Accordingly, the efficiency of generating protons for hindering growth of tumor cells or necrotizing tumor cells may be improved. As a consequence, the treatment apparatus using protons according to the embodiment may be able to treat a tumor site more effectively.

FIG. 3 is a sectional view to explain the proton generating target material according to the embodiment of the present invention and a manufacturing method thereof.

FIG. 3 shows the metal film 212 which may include a high-conductivity material such as Au or Al.

The H-containing film 214 is disposed on one surface of the metal film 212. The H-containing film 214 may be formed by chemical vapor deposition (CVD). According to an exemplary embodiment, the H-containing film 214 may be formed by plasma enhanced CVD (PECVD). Here, the H-containing film 214 may be an a-Si:H film and also may be formed in thickness of about 10 to 500 Å.

The a-Si:H film may be formed by performing the CVD with respect to the surface of the metal film 212 using a gas including SiH₄, Ar, and H₂. The hydrogen atom content of the a-Si:H film may be adjusted according to conditions of the CVD. The conditions of the CVD may include composition of the gas, pressure, CVD temperature, and processing time. For example, the hydrogen atom content of the a-Si:H film may increase as a hydrogen content in the gas is higher, the pressure is lower, the temperature is lower, and the processing time is longer. Thus, the number of hydrogen atoms of the a-Si:H film may be adjusted relative to the number of silicon atoms according to variation of the conditions of the CVD.

The proton generating target material according to the above-described embodiment is provided with a film having a quantitative hydrogen content so that protons are generated in a specific quantity required for a tumor site to be treated. Therefore, a proper quantity of the protons may be projected to the tumor site of the patient. Accordingly, the efficiency of generating protons for hindering growth of tumor cells or necrotizing tumor cells may be improved. As a consequence, the proton generating target material according to the embodiment may be more effectively used in treating a tumor site.

Furthermore, in the proton generating target material generated by the method according to the embodiment of the present invention, the film having the quantitative hydrogen content to generate the specific quantity of protons is manufactured through a simple deposition process. As a result, manufacturing cost of the proton generating target material is reduced and cost for treating tumor may accordingly be reduced.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A target material for generating protons, comprising:
a supporting film; and
a hydrogenated amorphous silicon (a-Si:H) film disposed on the supporting film.

2. The target material of claim 1, wherein the a-Si:H film has a quantitative hydrogen atom content.

3. The target material of claim 1, wherein the a-Si:H film is provided on the supporting film by chemical vapor deposition (CVD).

4. The target material of claim 1, wherein the supporting film comprises a conductive material.

5. The target material of claim 4, wherein the conductive material comprises gold (Au) or aluminum (Al).

6. A method of manufacturing a target material for generating protons, the method comprising:
preparing a supporting film; and
forming a hydrogenated amorphous silicon (a-Si:H) film on the supporting film.

7. The method of claim 6, wherein forming the a-Si:H film is performed by chemical vapor deposition (CVD).

8. The method of claim 7, wherein forming the a-Si:H film is performed by plasma enhanced CVD (PECVD).

9. The method of claim 7, wherein a hydrogen atom content of the a-Si:H film is adjusted according to conditions of the CVD.

10. The method of claim 9, wherein the conditions of the CVD comprise composition of gas, pressure, deposition temperature, and processing time.

11. The method of claim 6, wherein the supporting film comprises a conductive material.

12. The method of claim 11, wherein the conductive material comprises gold (Au) or aluminum (Al).

13. A treatment apparatus using protons, comprising:
a cylindrical bore member having an inner space to receive a patient;
a proton generating target material provided at an inner surface of the bore member; and
a laser adapted to supply a laser beam to the proton generating target material so that protons are generated from the proton generating target material and projected to a tumor site of the patient,
wherein the proton generating target material comprises a supporting film and a hydrogenated amorphous silicon (a-Si:H) film provided on the supporting film.

14. The treatment apparatus of claim 13, wherein the a-Si:H film has a hydrogen atom content of a specific percentage for treatment of the tumor site of the patient.

15. The treatment apparatus of claim 13, wherein the a-Si:H film is provided on the supporting film by chemical vapor deposition (CVD).

16. The treatment apparatus of claim 13, wherein the supporting film comprises a conductive material.

17. The treatment apparatus of claim 16, wherein the conductive material comprises gold (Au) or aluminum (Al).

18. The treatment apparatus of claim 13, wherein the inner space of the bore member is maintained under vacuum.

19. The treatment apparatus of claim 13, wherein the laser is a high-output laser.
